(19) United States
(12) Patent Application Publication
(10) Pub. No.: US 2026/0009788 A1
(43) Pub. Date: Jan. 8, 2026

(54) Method for Quantifying a Nucleic Acid Solution and Microfluidic Analysis Device

(71) Applicant: Robert Bosch GmbH, Stuttgart (DE)

(72) Inventors: Franz Laermer, Weil Der Stadt (DE); Samir Kadic, Langenau (DE); Hannah Bott, Straubenhardt (DE); Christian Grumaz, Waiblingen (DE); Sebastian Pilsl, Stuttgart (DE); Anne Hoffmann, Herrenberg (DE)

(21) Appl. No.: 18/992,783

(22) PCT Filed: Jun. 22, 2023
(86) PCT No.: PCT/EP2023/066940

§ 371 Date: Jan. 9, 2025

(30) Foreign Application Priority Data
Jul. 13, 2022 (DE) 10 2022 207 161.1

(51) Int. Cl. G01N 33/53

(52) U.S. Cl. CPC G01N 33/5308

(57) ABSTRACT

A method for quantifying a first solution containing at least one nucleic acid includes thinning the first solution with a second solution containing at least one intercalating fluorescent dye, carrying out a fluorescence measurement on the first solution in order to obtain a fluorescence signal, carrying out a specified number of repetitions of the two preceding steps, plotting the fluorescence signals against a thinning of the first solution, fitting a function to the fluorescence signals, determining a local maximum of the function or its first derivative, and determining a concentration of the nucleic acid from a fluorescence signal positioned before the local maximum.

The present invention relates to a method for quantifying a solution containing at least one nucleic acid. Furthermore, the present invention relates to a microfluidic analysis apparatus configured in order to carry out the steps of the method.

### PRIOR ART

For quantification of DNA by means of intercalating fluorescent dye, a defined amount of the fluorescent dye is added to the DNA solution to be measured, the emission of which increases significantly after intercalation or binding of nucleic acids, and then the fluorescence intensity is measured. Commonly used fluorescent dyes in this context are ethidium bromide, propidium iodide, DAPI (4′,6-diamidino-2-phenylindole), cyanine dyes, or cyanine-based dyes. The measured value is compared to an equally measured standard, and the previously unknown amount of DNA is determined thereby. In order to ensure that the measurement is made in a concentration range in which there is a linear association between the DNA concentration and the fluorescence intensity, the maximum and minimum amount of DNA that can be detected by the method is previously defined. When measuring an overly concentrated sample, an upper limit of fluorescence intensity is established. If this limit has been exceeded, the intensity measurement can no longer be used in order to directly infer the concentration, because it falls out of the linear measurement range.

EP 3 266 880 A1 describes a fluorescence-based quantification of a target nucleic acid using a set of a quantitative amplification of reference samples and a set of quantitative amplification of the target nucleic acid. A reference table is generated from the data set of the reference samples, which is used in order to quantify the target nucleic acid. The data sets each describe a curve that is fitted with a sigmoidal function.

### DISCLOSURE OF THE INVENTION

In the method for quantifying a first solution containing at least one nucleic acid, in particular DNA, the first solution is first thinned with a second solution containing at least one intercalating fluorescent dye. An intercalating fluorescent dye is understood in particular to mean a fluorescent dye whose emission increases after intercalation or binding of nucleic acids. The fluorescent dyes used can be in particular ethidium bromide, propidium iodide, DAPI (4′,6-diamidino-2-phenylindole), cyanine dyes, or cyanine-based dyes. This not only reduces the concentration of nucleic acid in the first solution, but it is also contacted with the fluorescent dye so that it can intercalate or bind to the nucleic acid. Mechanisms that lead to such behavior can be: Stiffening of the molecular structure as a result of bonding to the nucleic acid backbone, thereby creating less vibrational relaxation and higher fluorescence quantum yields and/or charge induction and greater polarization through coupling to nucleic acids (negatively charged nucleic bases), thereby inducing higher dipole torques for optical transitions, having the result of greater absorption and fluorescence (transitional matrix elements). Thereafter, a fluorescence measurement is carried out on the first solution in order to obtain a fluorescence signal. The steps of thinning and carrying out the fluorescence measurement are repeated several times, wherein the number of repetitions is specified. Thus, after each thinning, a fluorescence measurement is carried out on a less concentrated solution of the nucleic acid. Because the concentration of the nucleic acid is not yet known, the obtained fluorescence signals cannot yet be assigned to concentrations of the nucleic acid, but rather initially only to thinning steps.

After the specified number of thinnings and fluorescence measurements have been carried out, the fluorescence signals are plotted against a thinning of the first solution. Thus, a first thinning step, a second thinning step, and so forth are plotted on the x-axis, whereas the fluorescence intensities to be assigned to the respective thinning step are plotted on the y-axis. A function is then fitted to the fluorescence signals. This function can be in particular sigmoidal, polynomial, exponential/potential, or logarithmic. A local maximum of the function or a local maximum of a first derivative of the function is determined. In particular, the local maximum closest to the highest thinning step is to be selected. This is hereinafter referred to as the relevant local maximum. A concentration of the nucleic acid is then determined from a fluorescence signal that is positioned before the local maximum, in particular before the relevant local maximum. “Before” in this context means that the thinning step of the fluorescence signal used in order to determine the concentration is higher than the thinning step of the local maximum. The determination can in particular be carried out by comparison to an equally measured standard.

This method allows an intrinsic control over which thinning step is optimally positioned for quantification in the linear range of the relationship between nucleic acid concentration and fluorescence intensity. The occurrence of a local maximum, in particular a relevant local maximum, of the function serves as an indicator for detecting that the linear range is being exited. The method is thus suitable for quantifying a solution containing a nucleic acid in an automated, in particular microfluidic, system in which a manual thinning and subsequent re-measuring of the solution is not possible. This goal could not be achieved in a simpler manner by establishing a maximum fluorescence intensity from which an exit from the linear range is to be assumed, because there could be lower measured values due to interference of some fluorescent dyes, for example fluorophores.

The steps of thinning and carrying out the fluorescence measurement are preferably repeated at least four times each. This ensures that sufficient fluorescence signals are available in order to, on the one hand, fit the function and, on the other hand, find a measured value suitable for determining the concentration below the local maximum.

The determination of the concentration is preferably made from a fluorescence signal, which is positioned at least two thinning steps before the local maximum, in particular before the relevant local maximum. A further preferable determination of the concentration can also be carried out at the thinning step closest to the point having the highest positive slope before the local maximum, in particular before the relevant local maximum. When using the fluorescence signal positioned directly before the local maximum, in particular before the relevant local maximum, there is a risk that it already lies outside the range of a linear relationship between the concentration of the nucleic acid and the fluorescence intensity. A fluorescence signal even further upstream of the local maximum would already have an unnecessarily high thinning.

In a preferred embodiment of the method, the local maximum of the function, in particular the relevant local maximum, is determined from a first derivative of the function. In a further preferred embodiment of the method, the local maximum of the first derivative of the function, in particular the relevant local maximum, is determined from the second derivative of the function.

If it turns out that the function and its first derivative has no local maximum, in particular no relevant local maximum, then all measured fluorescence signals lie in the range of a linear association between nucleic acid concentration and fluorescence intensity. In this case, it is preferred that the concentration is determined from the fluorescence signal of the first carrying out of the fluorescence measurement, because the first solution is still least thinned in this measurement.

The method can be used in order to operate a microfluidic analysis apparatus configured so as to carry out the steps the method. For this purpose, the microfluidic analysis apparatus comprises a structural means for being able to carry out the steps of thinning and carrying out the fluorescence measurement repeatedly. On the other hand, it comprises means for plotting the fluorescence signals against the thinning of the first solution, fitting a function to the fluorescence signals, determining a local maximum of the function, and determining a concentration of the nucleic acid from the fluorescence signal positioned before the local maximum. For this purpose, these method steps are implemented in particular as a computer program.

### BRIEF DESCRIPTION OF THE DRAWINGS

Exemplary embodiments of the invention are illustrated in the drawings and explained in further detail in the following description.

FIG. 1 schematically shows a portion of a microfluidic analysis apparatus according to one exemplary embodiment of the invention.

FIG. 2 shows a flowchart of an exemplary embodiment of the method according to the invention.

FIG. 3 shows in a diagram an association between a concentration of DNA and a fluorescence intensity in an exemplary embodiment of the invention.

FIG. 4a shows in a diagram a polynomial function, which reflects an association between a concentration of DNA and a fluorescence intensity in an exemplary embodiment of the invention.

FIG. 4b shows in a diagram the first derivative of the polynomial function according to FIG. 4a.

### EXEMPLARY EMBODIMENTS OF THE INVENTION

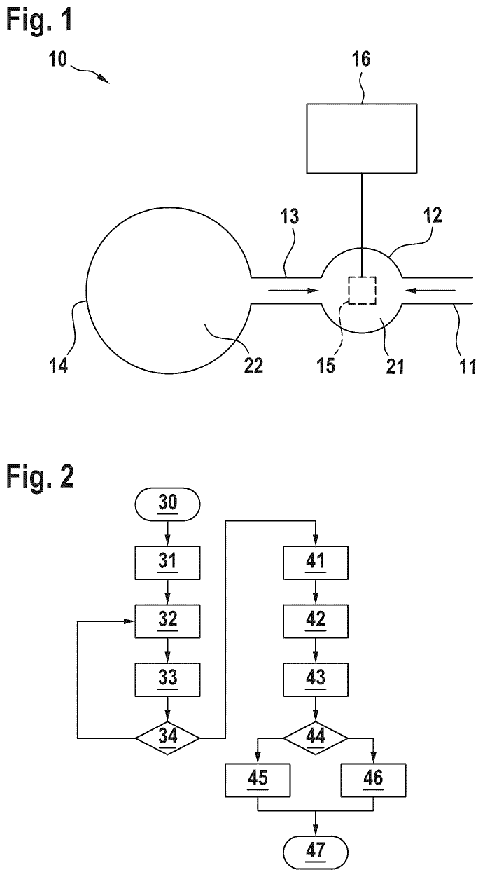

FIG. 1 shows elements of a microfluidic analysis apparatus 10 according to an exemplary embodiment of the invention. These elements are partially arranged in a fluidic layer of a microfluidic cartridge and partially arranged in an analysis unit that receives the microfluidic cartridge. A first microfluidic channel 11, which opens up into a first microfluidic chamber 12, is arranged in the fluidic layer. It is configured so as to transport a first solution 21 into the first microfluidic chamber 12. The first solution 21 is an aqueous solution of DNA in a buffer medium. The concentration of DNA is unknown. Via a second microfluidic channel 13, the first microfluidic chamber 12 is connected to a second microfluidic chamber 14. In the second microfluidic chamber 14, a second solution 22 is stored. The second solution 22 contains an intercalating fluorescent dye (for example, contained in the Qubit™ 1×dsDNA HS Working Solution by Thermo Fisher Scientific). The first microfluidic chamber 12 comprises a transparent window, above which a fluorescence sensor 15 of the analysis unit is arranged. The fluorescence sensor 15 comprises a light source in order to excite fluorescence in the first solution 21 and a sensor in order to detect the fluorescent response. It is connected to an electronic computational device 16, which is also connected to a pneumatic manifold (not shown) of the analysis unit. A pneumatic layer of the cartridge can be actuated via the pneumatic manifold in order to control fluid flows in the fluidic layer.

The sequence of a method according to an exemplary embodiment of the invention is shown in FIG. 2. The latter is implemented as a computer program in electronic control unit 16. After the start 30 of the method, the first solution 21 is first placed 31 in the first microfluidic chamber 12, in that it is pumped into the first microfluidic channel 11. Then, a thinning 32 of the first solution 21 with the second solution 22 is carried out, wherein the thinning ratio in the present exemplary embodiment is 1:2. The thinning occurs in that a portion of the second solution 22 stored in the second microfluidic chamber 14 is pumped through a second microfluidic channel 13 into the first microfluidic chamber 12. Subsequently, a fluorescence measurement is carried out 33 on the first solution 21 by means of the fluorescence sensor 15. The method steps 32 and 33 are repeated until a test 34 shows that six thinnings of the first solution have been carried out and six fluorescence signals have been received.

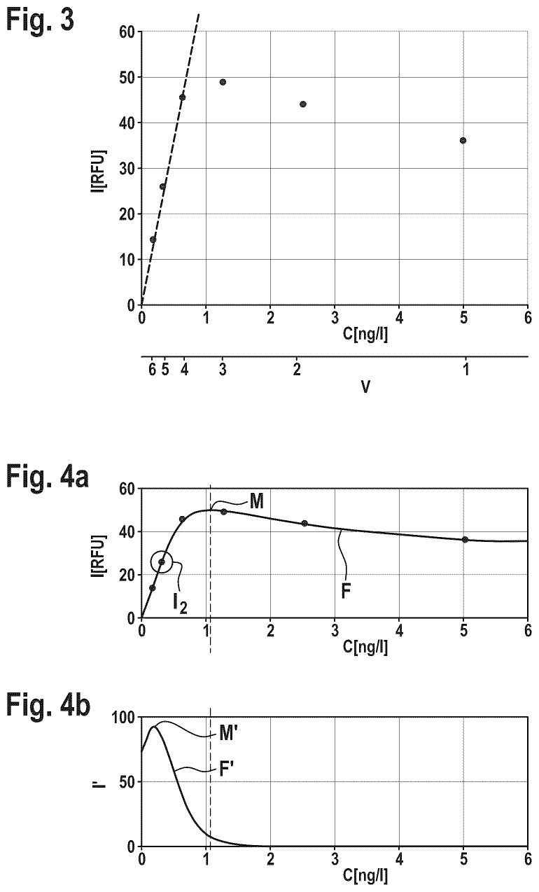

This is followed by the plotting 41 of the fluorescence signals against a thinning of the first solution. FIG. 3 shows in a diagram the association between the concentration C of DNA in the first solution 21 and the measured fluorescence intensities I in relative units (RFU=relative fluorescence unit). It can be seen that, only for the three lowest concentrations, there is a linear association between concentration C and fluorescence intensity I. Because the concentration C is not yet known, the plotting is instead carried out against thinning steps. The largest concentration C of 5 ng/l shown in FIG. 3 corresponds to the first thinning step, and each subsequent halving of the concentration C corresponds to a further thinning step.

A fitting 42 of a polynomial function F to the fluorescence signals is now carried out. The fitted function F is shown in FIG. 4a. This is followed by a determination 43 of a local maximum M of function F, in that the first derivative F′ of function F is calculated. This is shown in FIG. 4b. By means of a dashed line connecting FIG. 4b to FIG. 4a, the position of the maximum M in the function F is shown in FIG. 4a. Alternatively, a local maximum M′ of the first derivative F′ of the function F′ is calculated. This can be done by forming the second derivative.

Then, there is a test 44 of whether a local maximum M or M′ has been found. If this is the case, as shown in FIGS. 4a and 4b, then there is a determination 45 of the concentration C from the fluorescence signal I₂ positioned before the local maximum M or M′. The relationship between fluorescence intensity I and concentration C is established by a reference measurement. A chamber with the reference sample is also arranged in the fluidic layer of the microfluidic cartridge, but not shown in FIG. 1.

If the test 44 shows that no local maximum M or M′ has been found, then a determination 46 of the concentration C is made from the fluorescence signal of the first carrying out 33 of the fluorescence measurement.

After the concentration C has been determined by one of the method steps 45 or 46, the method is ended 47.

1. A method for quantifying a first solution containing at least one nucleic acid, comprising:
thinning the first solution with a second solution containing at least one intercalating fluorescent dye;
carrying out a fluorescence measurement on the first solution in order to obtain a fluorescence signal;
carrying out a specified number of repetitions of the thinning of the first solution and the carrying out of the fluorescence measurement;
plotting the fluorescence signals against a thinning of the first solution;
fitting a function to the fluorescence signals;
determining a local maximum of (i) the function or (ii) the first derivative of the function; and
determining a concentration of the nucleic acid from one of the fluorescence signals occurring before the local maximum.

2. The method according to claim 1, wherein the specified number of repetitions includes at least four repetitions.

3. The method according to claim 1, wherein the determination of the concentration includes determining the concentration of the nucleic acid from one of the fluorescence signals occurring at least two thinning steps before the local maximum.

4. The method according to claim 1, wherein the local maximum of the function is determined from the first derivative of the function.

5. The method according to claim 4, wherein the local maximum of the first derivative of the function is determined from the second derivative of the function.

6. The method according to claim 1, wherein the determination of the concentration includes determining the concentration from the fluorescence signal of the first carrying out of the fluorescence measurement when the function and its the first derivative of the function do not have a local maximum.

7. A microfluidic analysis apparatus, comprising:
a first microfluidic chamber;
a fluorescence sensor; and
a computational device configured to:
thin a first solution containing at least one nucleic acid in the first microfluidic chamber with a second solution containing at least one intercalating fluorescent dye;
carry out a fluorescence measurement on the first solution with the fluorescence sensor to obtain a fluorescence signal;
carry out a specified number of repetitions of the thinning of the first solution and the carrying out of the fluorescence measurement;
plot the fluorescence signals against the thinning of the first solution;
fit a function to the fluorescence signals;
determine a local maximum of (i) the function or (ii) the first derivative of the function; and
determine a concentration of the nucleic acid from one of the fluorescence signals occurring before the local maximum.
